(19) United States
(12) Patent Application Publication
(10) Pub. No.: US 2021/0096123 A1
(43) Pub. Date: Apr. 1, 2021

(54) NUCLEIC ACID METHYLATION ANALYSIS

(71) Applicant: Haitao Huang, Camas, WA (US)

(72) Inventor: Haitao Huang, Camas, WA (US)

(21) Appl. No.: 17/037,267

(22) Filed: Sep. 29, 2020

Related U.S. Application Data
(60) Provisional application No. 62/908,070, filed on Sep. 30, 2019.

(51) Int. Cl. G01N 33/487; C12Q 1/6886; B01L 3/00; G01N 27/07

(52) U.S. Cl. CPC G01N 33/48721; C12Q 1/6886; B01L 2300/0645; G01N 27/07; C12Q 2600/154; B01L 3/502761

(57) ABSTRACT

This invention provides systems and methods for detecting the presence of cancer cell nucleic acids in a fluid sample. The fluid samples are applied to, e.g., a microfluidic cartridge input port to contact a surface that selectively adsorbs hypomethylated nucleic acids, which adsorption is detectable as, e.g., a change of impedance or vibration frequency at the surface.

### FIELD OF THE INVENTION

The inventions described herein involve systems and methods in the field of cancer detection. Nucleic acids low in methylation and/or presenting clustering of methylation tend to selectively adsorb onto surfaces of a sensor and can be detected by resultant changes in the impedance or vibration frequency at the sensor. Systems include fluidic cartridges providing a sample fluid to contact impedance electrodes or piezoelectric sensors. The cartridges interact with assay devices that actuate the cartridges and detect the sensor output.

### BACKGROUND OF THE INVENTION

Cancer detection often depends on disease progression to the point where a patient is expressing unpleasant symptoms. Often, this results in missed opportunities for effective early intervention treatments. Certain cancers can be detected by general physical examination procedures, such as palpation for nodules. Routine screening of blood samples can identify specific cancer indicators such as cancer-associated antigens or the presence of immature cells in a blood cell differential review. However, in these cases, the cancer has typically existed in the body for many months.

In Nature Communications 9: 4915, by Sina, et al., it is noted that most neoplastic cells include many epigenetic modifications of their nucleic acids. For example, the genomic nucleic acids of many cancer cells exhibit a substantial reduction of methylation. In addition to reduced methylation, there exists a patterning of nucleic acid methylation in clusters in a “methylation landscape” between large tracts of relatively hypomethylated regions. These molecular features of the nucleic acids can influence macro-scale phenomena through their altered affinity for surfaces, aggregation conditions, and electrical resistance. Sina has detected nucleic acids of cancer cells by electron microscopy, surface adhesion morphology, differential aggregation, and differential pulse voltammetry (DPV).

We believe the present techniques of detecting cancer nucleic acids are problematic. They typically require at least two process steps, adsorption steps in the sample before transfer to detection, the use of complex reagents, and/or human eye detection and interpretation. Further the output is typically only qualitative. In view of the above, a need exists for a simple generic assay for cancer. Such an assay should use a small patient sample and not require substantial processing by a technician. We believe benefits could also be realized through application of methods in micro-scale cartridges. The present invention provides these and other features that will be apparent upon review of the following.

### SUMMARY OF THE INVENTION

Systems and methods are presented for detecting hypomethylated/clustered nucleic acids in fluids associated with cancer cells, e.g., by monitoring changes in impedance or vibration frequency when the nucleic acids are selectively adsorbed onto sensor surfaces. The sample fluids can include, e.g., whole blood, plasma, isolated nucleic acids in solution, a cell lysate, and/or the like. The systems typically include, e.g., a fluidic cartridge received within, and in electric contact with, a detector device. The cartridge has fluidic channels through which a sample fluid can flow to contact sensor surfaces adapted to adsorb hypomethylated/clustered nucleic acids, associated with the presence of cancer cells. Adsorbed nucleic acids change the impedance or vibration frequency sensor elements, which can be detected and analyzed by the detector device. Methods of detecting the hypomethylated/clustered nucleic acids include application of a sample fluid into the cartridge, adsorption of nucleic acids onto the detector surfaces, monitoring of any changes to the sensor output signal, and analysis of output signal to determine the presence or quantity of hypomethylated/clustered nucleic acids in the sample.

The systems can include a cancer cell nucleic acid detection system comprising a cartridge and a detector device. The cartridge can include a sample input port leading to paired detector electrodes through a first microfluidic channel The electrodes are adapted to adsorb nucleic acids and are in electric contact through conductors to contact pads on an outer surface of the cartridge. The detector device has an AC power source and impedance detector in contact with the cartridge contact pads and configured to measure impedance (and/or resistance) between the electrodes. With this arrangement, hypomethylated nucleic acids associated with the cancer cell can adsorb onto one or more surfaces of the detector electrodes with higher affinity than nucleic acids of non-cancer cells, e.g., resulting in a higher AC impedance for samples having cancer cell nucleic acids than for normal samples not containing cancer cell nucleic acids.

The cartridge is typically a microfluidic cartridge (e.g., with flows driven by capillary action). The electrodes typically comprise a gold, platinum, or hydrophobic surfaces, e.g., located on opposite sides of a cartridge channel or chamber. In certain cases, a channel or vented waste chamber is positioned downstream from electrodes. The system is configured to have applied voltage of 0.01 V-5 V, with preferred 1 V of, e.g., and/or a frequency is 10 Hz-500 KHz with preferred at 1K. For example, in some embodiments the output to electrodes in use can be about 1 V at about 1K Hz and have current in the range of 2-100 micro amps. The cartridge can further comprise a second channel configured to provide a confirmatory or control assay branch.

In some embodiments, the cartridge may contain one or more reagents. For example, a cartridge channel or chamber can contain a depot of a dried buffer reagent that enhances selective aggregation/adsorption conditions for hypomethylated nucleic acids. The buffer can comprise, e.g., a pH buffer, surfactants, lysing agents, conductive ions, enzyme, and/or the like.

The system can be configured to detect cancer cell nucleic acids with a methylation landscape of hypomethylated nucleic acids having a methylation values of between 30% and 50% and/or methylation clustering non-uniformly with most methylation within 500 bp of another methylation site on the same nucleic acid molecule.

In the system, the detector can be configured to distinguish the presence of the cancer cell nucleic acids when the detected impedance between the electrodes is 10% more than for negative control sample. The negative control can be, e.g., a normal nucleic acid control, e.g., from the same species or from patient's cells known not to be cancerous. In a preferred embodiment, the system is configured so that the hypomethylated nucleic acids aggregate in a layer or film on the detection electrode surface without a prior aggregation step in solution.

In an alternate embodiment, the device sensor is an oscillated quartz crystal with a gold surface exposed to the inside of the microchannel. For example, the cancer cell nucleic acid detection system can include a microfluidic cartridge and a detector device. The cartridge can have a sample input port leading through a first microfluidic channel to a piezoelectric quartz crystal sensor having a gold surface, the sensor adapted to adsorb nucleic acids (e.g., onto a gold surface). Conductors lead from the sensor to contact pads on an outer surface of the cartridge. The detector device has an oscillator circuit output configured to stimulate a base frequency in the crystal through the contact pads and conductor. The crystal vibration frequency is detected by a frequency counter detector in contact with the cartridge contact pads and configured to measure a frequency of crystal vibrations. When hypomethylated nucleic acids associated with the cancer cell adsorb onto the crystal gold surface the vibration frequency of the crystal is slowed, and the lower frequency is detected by the frequency counter.

In a typical arrangement, the oscillator circuit frequency is tuned to provide series or parallel resonant vibrations (Fs or Fp) in the range from 1 MHz to 100 MHz (e.g., 1 Hz to 50 MHz, 2.5 MHz to 10 MHz, or about 5 MHz) at the crystal when nucleic acids are not adsorbed (e.g., when the crystal surface is in contact with pure water). The presence of a 30% to 50% methylated nucleic acid (e.g., hypomethylated relative to an associated non-cancer cell nucleic acid) can be confirmed, e.g., on detection of a when a drop of Fs or Fp in the crystal oscillation, ranging from 10 Hz to 1000's Hz.

The hypomethylated/clustered nucleic acids can be adsorbed onto the gold surface by adjustment of the aqueous solvent environment of the sample. For example, the ionic strength and/or pH of the sample can be adjusted by reagents in the microchannels of the cartridge. The cartridge can include depot of a dried reagent adapted to enhance adsorption of the hypomethylated nucleic acid onto the sensor surface. The dried reagent can include, e.g., a buffer, a surfactant, a lysing agent, salts, and/or the like. For example, the dried reagent can comprises a sodium salt adequate to provide the volume of sample flowing past the depot with a concentration of from 200 mM to 1 M of the salt, e.g., at a near neutral pH. The dried buffer can be located in a chamber between the input port and crystal sensor, wherein the chamber has a cross-section across fluid flow at least 50% greater than the first microfluidic channel. Optionally, the sample can be adjusted before application to the cartridge input port.

The systems of the invention can be configured to include one or more additional channels, e.g., configured to provide a confirmatory or control assay. For example, as shown in FIG. 1B, the first channel 12 and an identical second channel 16 can receive sample fluid through a shared input port 11; so that if the data results from the two channels are significantly different, the results can be considered suspect. Optionally, a second channel can be a negative control (without reagents or receiving a different fluid, e.g., without any hypomethylated nucleic acids).

The systems can be adapted to selectively detect hypomethylated nucleic acids. For example, the detector surfaces, detector electronics, sample contact dwell time, and sample volume can be adjusted to optimize for a desired sensitivity, specificity, precision of results, sample type, or time per assay. The assays can be optimized (theoretically or empirically) to adsorb of nucleic acids with 30% to 50% methylation over other nucleic acids. For example, the ionic strength of the sample solution can be adjusted, e.g., taking advantage of the fact that hypomethylated cancer-associated nucleic acids adsorb first as ionic strength of the sample is increased. The system can be tuned to detect cancer cells with a methylation landscape of hypomethylated nucleic acids having a methylation values from 30% to 50% or methylation clustering with more than half the methylated cysteines represented in 20% or less of the nucleic acid. The system can be configured so that the hypomethylated nucleic acids aggregate on the crystal sensor surface without a prior aggregation step in solution.

The systems and devices of the inventions can be used in methods for detecting cancer-associated nucleic acids in fluid samples. For example, the methods of detecting the presence of a cancer in a patient can include providing a microfluidic cartridge comprising a sample input port, a first microfluidic channel, and a pair of electrodes; wherein the a fluid sample applied to the input port flows by capillary action through the channel to the pair of electrodes. A detection device can have an AC current power supply and an impedance detector. A patient biological fluid sample is applied to the input port to flow through the channel, coming in contact with the sensor electrode pair. The AC current is applied across the electrode pair from the power supply and impedance between the paired electrodes is detected by the detector device. If the sample contains more hypomethylated DNA than for a sample without hypomethylated DNA, this will be detected as relatively high impedance across the sensor electrodes, e.g., due to resistive coating of the electrode surface with hypomethylated (and/or clustered) cancer cell nucleic acids on the sensor surface. DNA's of raw samples, such as blood or tissue may need to be extracted either in a separate apparatus or included with the device.

The method can be configured to detect impedance values at a time point, monitor impedance over time, and/or monitor rates of impedance change. The fluid sample can flow into a detection chamber to contact the electrodes and stop, or the fluid sample can flow across the electrodes to a waste chamber, e.g., to accumulate sample contact with the electrodes. In one embodiment, the presence of hypomethylated nucleic acid is determined when the measured impedance increases by 10 to 20 percent within 10 seconds of contact of the electrode pair with the fluid sample. In another embodiment, the presence and/or quantity of the hypomethylated nucleic acid is evaluated with reference to a sigmoid curve of impedance over time (e.g., based on the steepest point or half height on the curve). The presence of cancer cell nucleic acid can be confirmed, e.g., when the percent methylation is within the range of from 30% to 50% and/or when more than 50% of the nucleic acid methylations are present within less than 20% of the sample gene sequences. In a preferred embodiment, the electrodes and detector are configured to detect less than 5 pg of cancer nucleic acid in a sample and/or to detect cell free cancer nucleic acids present at a level of less than 1% of total cell free nucleic acids. Detection of cancer nucleic acids according to the methods can be sensitive to a combination of hypomethylation and methylation clustering character of the adsorbed nucleic acids.

A second channel can be provided on the cartridge with a second electrode pair. The second channel can receive a duplicate sample, a control sample, or a reference sample fluid at a second electrode pair. The presence or quantity of hypomethylated nucleic acid can be determined and/or the assay value validated by comparison to the reference impedance at the second electrode pair. The presence of hypomethylated nucleic acids can be indicated when impedance for a sample rises above a pre-established cut-off impedance value. The cut-off value can be determined, e.g., empirically by review of reference samples of known hypomethylation/clustering character. Detecting cancer cell nucleic acids can be by impedance, without reference to redox potential measurement, amperometry, or imaging of the electrodes.

In another aspect of the invention, the sensor is a piezoelectric feature having an affinity surface interacting with methylated nucleic acids. For example, a cancer cell nucleic acid detection system can include a cartridge and a detector device, wherein the cartridge has a sample input port leading through a first microfluidic channel to a piezoelectric quartz crystal sensor having an affinity surface, the sensor adapted to adsorb nucleic acids; and conductors leading from the sensor to contact pads on an outer surface of the cartridge. The affinity surface can be a noble metal or a bioaffinity molecule coating. The detector device includes an oscillator circuit output configured to stimulate a base frequency in the crystal. A frequency counter detector is also in contact with the cartridge contact pads and configured to measure a frequency of crystal vibrations. When hypomethylated nucleic acids associated with the cancer cell adsorb onto the surface with higher affinity than nucleic acids of non-cancer cells, their presence is detected by the frequency counter as a lower frequency from the crystal.

The piezoelectric cartridge system can be configured with the detector oscillator circuit frequency ranging from, e.g., 1 MHz to 100 MHz when the crystal is in contact with pure water. The system can be configured to confirm the presence of a 30% to 50% methylated nucleic acid when a 10 Hz to 1000's Hz drop in Fs or Fp of the crystal oscillation is detected.

As with the impedance configured system, the cartridge can have a depot of a dried reagent adapted to enhance aggregation of the hypomethylated nucleic acid. The dried reagent can include a sodium salt adequate to provide the volume of sample flowing past the depot with a concentration of from 300 mM to 1 M of the salt. A second channel can be configured to provide a confirmatory or control assay. The system can be adapted to detect cancer cells with a methylation landscape of hypomethylated nucleic acids having a methylation values from 30% to 50% or methylation clustering with more than half the methylated cysteines represented in 20% or less of the nucleic acid. The system can be configured so that the hypomethylated nucleic acids aggregate on the crystal sensor surface without a prior aggregation step in solution.

The inventions include methods of using piezoelectric sensors to detect hypomethylation. A method of detecting the presence of a cancer in a patient can include providing the piezo cartridge, applying a biological fluid sample to the input port wherefrom the fluid flows through the channel to contact the sensor surface, applying an AC current to the piezoelectric sensor from the power supply and monitoring a vibration frequency of the crystal with the frequency counter. In this way, the presence of hypomethylated nucleic acids can be indicated, e.g., when the monitored frequency is lower for the sample than for a sample without hypomethylated DNA.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” can include plural referents unless the content clearly dictates otherwise. Thus, for example, reference to “a surface” can include a combination of two or more surfaces; reference to “nucleic acids” can include mixtures of different nucleic acids, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein, the term “microfluidic” refers to systems or devices having a fluid flow channel with at least one cross sectional dimension less than 1000 μm. Most microfluidic channels allow capillary flow, e.g., depending on the affinity of a particular fluid for the channel walls. Some functional capillary scale channels of the invention can be greater than microfluidic scale. A microfluidic channel can have a cross-sectional dimension of 1000 μm, or less, 500 μm or less, 300 μm or less, 100 μm or less, 50 μm or less, or 10 μm or less. In many embodiments, the channel dimension is about 50 μm to 100 μm, but typically not less than 1 μm. Most microfluidic channels are capillary channels owing to their dimensions within the capillary geometry contact angles of typical liquids. This can, of course, depend on the affinity (e.g., contact angle) between the channel surface and the particular fluid. Capillary channels can have a least cross-sectional dimension of more than 1 mm, but this is less typical. Capillarity is a general term referring to phenomena attributable to the forces of surface or interfacial tension. A capillary scale chamber or channel has at least one dimension that functionally results in flow of an intended fluid along the chamber or channel surface by capillary action. Capillary scale chambers and channels of the invention can be at a microfluidic scale or not. Capillary flow can exist in channels with no microfluidic scale dimension, e.g., where the affinity for the fluid and surfaces are high, and/or the channel includes a porous material presenting smaller dimensioned topography. However, in the current inventions, the capillary scale dimension in the capillary channels is typically the height dimension, e.g., while the width dimension can be substantially larger.

A processing unit is an element of the assay device, e.g., that reads inputs from cartridges of the invention. The processing unit typically includes a digital computer, microcomputer, or integrated circuit, as understood in the art. The processing unit has electronic inputs to receive electronic parameters (e.g., from cartridge electrodes, piezoelectric crystals, interrogating photodiodes, etc.) or to receive data from accessory circuits (such as, e.g., user interface or cartridge information readers). The processing unit typically has output circuits, e.g., to actuate accessories, energize sensors, or update the user interface.

Impedance, as used herein is as understood in the field of electronics. For example, impedance is the effective resistance of an electric circuit or component to alternating current, arising from the combined effects of ohmic resistance and reactance.

Methylation is a process by which methyl groups are added to cysteines of a nucleic acid, typically at a CpG location. As used herein, a “hypomethylated” nucleic acid of a cell is methylated less than typical for that type of cell in a normal healthy condition. The nucleic acids of many normal cells are more methylated than the same type of cells after they are transformed into cancer cells. Hypomethylated nucleic acids from human cancer cells are typically not non-methylated, but less methylated than corresponding normal cells, resulting in, e.g., distinguishable aggregation/adsorption differences. For example, whereas a typical normal cell may be 60% methylated, cancer cells derived therefrom may be considered hypomethylated, e.g., at 45% methylation. As a general rule of thumb, a human nucleic acid can be considered hypomethylated if the nucleic acid is methylated in a range from 30% to 50% at CpG locations, or in a range of range from 30% to 50% as compared to methylation for the nucleic acid as found in non-cancerous cells of the same type from the same sample source.

Normal cells can be transformed into “cancer cells” when a series of mutations leads the cell to continue to grow and divide out of control. A normal cell, associated with a cancer cell, is a cell that is of the same differentiated type as the cell that was transformed into the cancer cell.

Methylation “clusters”, are regions on a nucleic acid rich in CpG sites that are methylated and clustered within a short span between larger relatively hypomethylated or unmethylated intergenic tracts. Methylation can be considered clustered, e.g., when more than half the methylated cysteines of a nucleic acid are represented in 20% or less of the nucleic acid length.

A “cartridge” is as known in the field of sample analyses. Typically, cartridges of the invention are microfluidic chips comprising channels, chambers, electrodes, adapted interact with a sample of and provide a signal correlated with a sample analyte of interest, as described herein. The cartridge is adapted to be functionally received in a contact with an assay device, e.g., to allow interactions with a processing unit energizing and receiving information from cartridge sensors.

A piezoelectric sensor is as is known in the art. For example, piezoelectric sensor responds to changes in physical pressure with a change in electrical charge. Typical piezoelectric sensors employ piezoelectric quartz crystals, e.g., in electrical contact with a voltage detector to monitor changes in pressure or vibration frequency of the sensor.

Reagents are as known in the art of clinical analysis. Reagents in the cartridges of the invention typically interact with samples to provide reaction conditions and/or to detectably interact (e.g., react or catalyze change) with analytes of interest in a sample. Reagents used herein provide, e.g., buffer and ionic strength conditions providing selective adsorption and/or aggregation of cancer cell nucleic acids over nucleic acids from associated normal cells.

Samples, in the context of the present assay devices and cartridges are typically liquids of interest containing one or more nucleic acid analytes of interest. Typical samples for analysis in the present cartridges can include, e.g., whole blood, plasma, other body fluids of an animal, and/or sample fluids from a manufacturing process.

As used herein, “substantially” refers to largely or predominantly, but not necessarily entirely, that which is specified.

The term “about”, as used herein, indicates the value of a given quantity can include quantities ranging within 10% of the stated value, or optionally within 5% of the value.

### BRIEF DESCRIPTION OF THE DRAWINGS

FIGS. 1A and 1B are schematic diagrams of exemplary cartridges using impedance probes for detection of hypomethylated nucleic acids.

FIG. 2 is a schematic diagram of a cartridge in a device for detection of hypomethylated nucleic acids.

FIG. 3 is a schematic diagram of a cartridge in a device for detection of hypomethylated nucleic acids using a piezoelectric sensor.

FIG. 4 is a schematic diagram of a simple functional impedance spectroscopy setup using a platinum wire electrode and gold disk electrode in a 2 mL centrifuge tube.

FIG. 5 is a schematic diagram of an exemplary microfluidic electrochemical impedance spectroscopy device.

FIGS. 6A and 6B show an example using a methylated DNA detection device based on monitoring changes of a quartz crystal resonant frequency.

### DETAILED DESCRIPTION

Described herein are methods and systems for detecting the presence of cancer-associated nucleic acids in a fluid. Typical cancer-associated nucleic acids are characterized as hypomethylated and/or presenting methylation in clusters separated by stretches of relatively non-methylated nucleic acids. A fluid sample is applied to the input port of a fluidic cartridge to flow in contact with paired electrodes adapted to monitor impedance or to contact a piezoelectric crystal sensor surface. Hypomethylated/clustered nucleic acids associated with the presence of cancer can be adsorbed onto the surfaces to alter an electronic parameter such as impedance between the electrodes or vibration frequency of the crystal. Changes in the electronic parameter, e.g., over values of normal samples without cancer-associated nucleic acids can indicate the presence of cancer in the sample source.

A number of methods and compositions are discussed in the Summary of the Invention and further details are provided herein and in the Examples section. As would be readily appreciated by the skilled person, the disclosures can be read in combination.

### Systems for Detecting Cancer-Associated Nucleic Acids

The systems for detecting the presence of cancer-associated nucleic acids in a fluid sample generally comprise a microfluidic cartridge and an associated electronic parameter monitoring device. For example, the cartridge can have in input port for a fluid sample leading to paired electrodes through a microfluidic channel. The electrodes are in electrical contact with contact pads on the exterior of the cartridge. When the cartridge is placed into the impedance monitoring device, the contact pads come into electric contact with device probes configured to measure impedance across the cartridge electrodes. The cartridge electrode surfaces tend to aggregate and/or adsorb sample nucleic acids to a greater degree if they are hypomethylated and/or presenting a clustered methylation landscape.

Fluid samples for analysis in the systems can be any of interest that may be suspected of containing nucleic acids from a cancer cell. For example, the fluid can be whole blood, plasma, serum, a cell or tissue lysate, CSF, urine, synovial fluid, an exudate, and/or the like. The sample can be unprocessed, or conditioned by, e.g., filtration, lysis, centrifugation, pH/ionic strength adjustment, and/or the like.

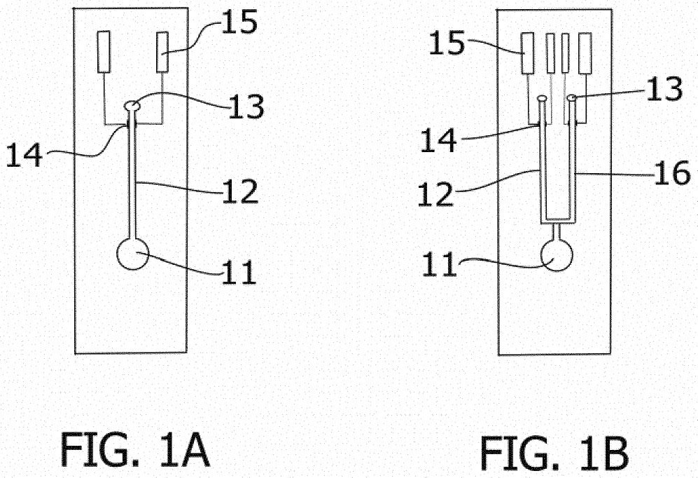

A basic cancer nucleic acid detection cartridge design can provide nucleic acid determinations and/or quantitative evaluation of methylation characteristics, e.g., using impedance electrodes along a single lateral flow channel. As shown in FIG. 1A, the nucleic acid detection cartridge includes a sample inlet port 11 in fluid contact through lateral flow channel 12 to vent 13. Impedance sensing electrodes 14 are in electrical contact with contact pads 15.

In a typical embodiment, the cartridge has a laminated design, including, e.g., top layer, middle channel/electrode layer, and bottom layer. The detection electrodes have surfaces in contact with flow channel fluids, wherein the surfaces are adapted to adsorb nucleic acids, particularly nucleic acids that are hypomethylated and/or which have clustered methylation.

The cartridges are typically thin, depending on the number of layers required. The cartridges can have a thickness (depth) ranging from more than 2 mm to less than 0.2 mm, from 1 mm to 0.3 cm, from 0.6 mm to 0.4 mm, or about 0.5 mm. A typical cartridge has laminated layers, though manufacture can be unitary, e.g., by molding, micro-machining, 3D additive manufacturing, and/or the like. The channel layer, containing the voids defining the channel cross sections are often the thinner layers, while the bottom (base) layer is often the thickest layer, and the top (cover) and any optional electrode layers are often intermediate in thickness. In one embodiment, the cover layer is about 175 um thick, the channel layer about 80 um, the electrode layer about 100 um, and the base layer about 250 um. The channel layer can have a thickness ranging from more than about 1 mm to less than about 0.04 mm, from 500 um to 60 um, or about 100 um. It is preferred that the cover and base layers be thicker than the channel layer, to provide the physical strength and minimize channel deformation, e.g., when the cartridge happens to be flexed. However, where conditions require the channel layer to be thicker, thinner overall depth can be retained by employing thinner base and cover layers.

The layers of the cartridge can be of the same material, or a combination of materials. The cartridge layers can comprise plastic, glass, metal, ceramic, and/or the like. However, the bulk of the cartridges and most typical layer materials are plastics. For example, polyethylene terephthalate (PET), polyethylene (PE), polyvinyl chloride (PVC), low-density polyethylene (LDPE), polypropylene (PP), polystyrene (PS), and the like. Due to its flexibility, inertness, and light transmission, a preferred plastic for flexible cartridge layers is PET. The assembled cartridge is configured to have a flexibility modulus of 1.5 GPa or less, 1 GPa, 0.5 GPA, 0.25 GPa, 0.1 GPA, or less.

The sensor surfaces are adapted to adsorb nucleic acids. As the nucleic acids accumulate (e.g., as a film or dispersion of aggregates) they can influence electronic parameters such as the capacitance, impedance, dielectric characteristics, and physical vibration frequency at the sensors. Changes in these parameters can be correlated and proportional to the amount of adsorbed nucleic acids. The surfaces can be adapted to selectively adsorb cancer nucleic acids over normal nucleic acids. For example, the electrodes can simply be a noble metal, such as rhenium, ruthenium, rhodium, palladium, silver, osmium, iridium, platinum, gold, and/or the like. These metals are resistant to oxidation and can have an affinity for nucleic acids, hypomethylated/clustered nucleic acids in particular. A preferred electrode metal surface is gold. Alternately, the electrode surface can be a metal modified by surface treatments, e.g., by anodizing, galvanizing, phosphatizing, enameling, blackening, electroplating, electro-polishing, electric dip-coat painting, chrome and nickel plating, plasma coating, and/or the like, to provide a surface selectively adsorbing cancer nucleic acids over normal nucleic acids to any significant or substantial degree. Optionally, the electrode surface may not be metallic, but include a film of organic material, such as a plastic or molecular film with selectivity for hypomethylated and/or clustered nucleic acids. Electrodes can have surfaces fabricated or treated to have a hydrophobicity optimized to preferentially adsorb hypomethylated and/or clustered nucleic acids, e.g., depending on the ionic strength of the sample fluid. Optionally, the electrodes can have affinity molecules, such as lectins or antibodies, selective for the cancer nucleic acids.

Optionally, the sensor in the channel can be a piezoelectric crystal, e.g., with a gold surface exposed to the channel interior. The piezoelectric sensor can receive an AC voltage from an oscillator circuit that establishes a certain vibration frequency in the sensor, e.g., in contact with an aqueous fluid. When the assay sample comes into contact with the sensor surface, hypomethylated nucleic acids (e.g., nucleic acids methylated in the range typically present in cancer cells) are adsorbed thereby adding mass and reducing the vibration frequency of the piezoelectric sensor. With the sensor vibrating at a high frequency, even a small change in mass can be detected with a high level of sensitivity and precision. The frequency of the piezoelectric sensor can be monitored by a frequency counter to detect and/or quantitate the influence of adsorbed nucleic acids.

The piezoelectric sensor can be a quartz crystal with a gold surface. For example, the sensor can be a cut thickness-shear-mode quartz crystal with gold electrodes, which is mounted in a cartridge microfluidic channel or chamber and in electrical contact with the computer-controlled frequency counter. The quartz crystal can be adapted for a steady operation in liquids at a frequency ranging from less than 0.1 MHz to more than 100 MHz, from 1 MHz to 50 MHz, or about 20 MHz. The presence of accumulated hypomethylated nucleic acids on the sensor surface can be interpreted qualitatively and/or quantitatively. For example, the presence of hypomethylated nucleic acids can be determined at a time point, or by monitoring a rate of sensor series or parallel resonant frequency (Fs or Fp) change. Sensitivity can be increased by allowing more time for adsorption and/or by allowing more sample to come into contact (larger diffusion chamber or a moving sample stream) with the sensor surface. In preferred embodiments, the presence of hypomethylated nucleic acids is detected in 20 seconds, 10 seconds, 5 seconds or less after sample fluid contact; or from less than 2 seconds to more than 120 seconds, or after 10 seconds. Optionally quantitation can be based on a standard time course, e.g., sigmoid curve half-height values.

The cartridge channels and chambers can include reagents that interact with the sample stream. For example, the channels can include reagents that provide a desired pH or ionic strength to the fluid. The pH and/or ionic strength of the fluid can be adjusted to enhance the ability to distinguish hypomethylated/clustered nucleic acids from normal nucleic acids according to the aggregation state and/or affinity for the electrode surface. The reagents can be, e.g., dry, gel, or liquid. Reagents can lyse cells from a sample to make the nucleic acids more available for interaction with the electrodes. Reagents can bring sample molecules into solution or out of solution, e.g., to reduce interference with the assay.

The cartridges can include filter elements to remove cells not of interest and/or aggregates from the fluid stream. For example, where nucleic acid of interest is a circulating cell-free nucleic acid, it can be desirable to remove cells or cell debris from the sample before interaction with reagents and/or electrode surfaces. Filters are typically located at or near the input port. The filters can be lateral flow or transverse flow.

The cartridge channels can be configured to terminate or cease flow at the electrodes, or can continue to flow by the electrodes. The channels can end in vents that allow for gas displacement, but stopping fluid flow at the electrodes. Alternately, the channels can continue on past the electrodes, e.g., allowing electrode surface contact with a larger volume of sample fluid and accumulating adsorbed nucleic acids. The channel can terminate with a vented waste chamber.

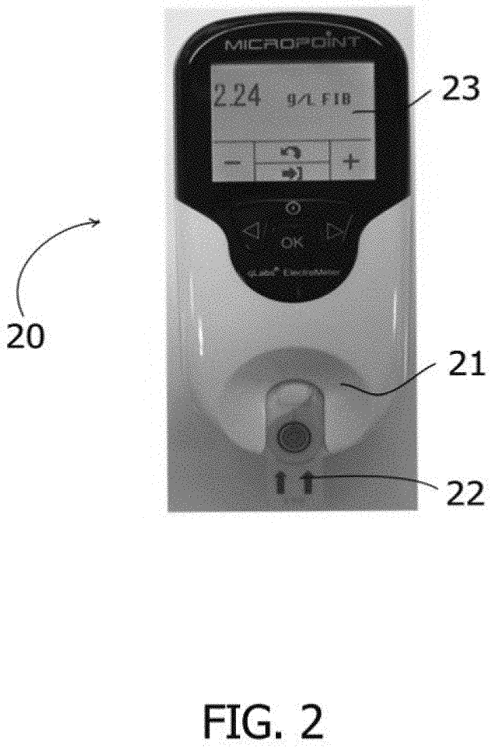

The present systems include the assay cartridges described herein functionally interacting with a detector device. The detector device can hold the cartridge, providing necessary electrical contacts between the cartridge and processing unit (e.g., digital computer), e.g., for receiving assay parameters, controlling outputs, and detecting signals from the cartridge. The assay device 20, e.g., as shown in FIG. 2 can be a hand held device with a docking area 21 receiving a cartridge 22. The assay device can have a user interface 23, e.g., for user inputs to the processing unit and processor output of assay results.

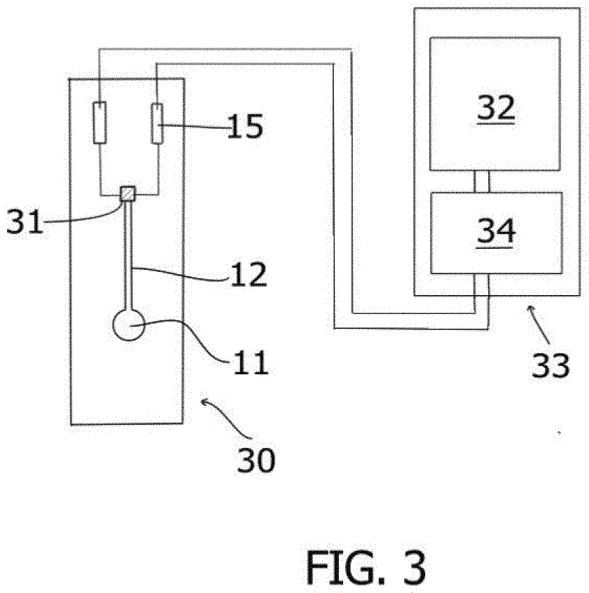

The processing unit can receive a variety of inputs. For example, the processing unit can receive user instructions from the user interface, and it can receive information (e.g., sample ID, patient ID, test type, cartridge type) from an inserted cartridge, as shown in FIG. 3. The processing unit 33 can be in electrical contact with the cartridge 30 to receive electrical inputs (e.g., resistance, amperage, capacitance, voltage, impedance, frequency) from cartridge sensors 31 contacting fluids during an assay. For example, the processing unit can be in contact with channel electrodes or piezo features having nucleic acid adsorption surfaces influencing impedance or frequency. In some embodiments, impedance detector of the processing unit has a sensitivity ranging from 0.1 μ [symbol] to 100 μ [symbol] .

The processing units can control outputs to energize the electrodes, environment controls, and detector components. The processing unit is typically digital and can receive digital input, e.g., from accessory sensors (e.g., having analog to digital convertors) in contact with cartridge electrodes or piezo crystals. For example, the processing unit can receive data from temperature sensors and be capable of sending output instructions (e.g., to a thermoelectric device) to maintain a programmed temperature for an assay. The processing unit can instruct application of a desired voltage and frequency to cartridge electrodes. The processing unit can output information requests or data (e.g., assay result) output to the user interface 32.

The processing unit can carry out, e.g., sequential steps of assay processes. The processing units can be programmed to carry out required inputs and outputs in the desired order. The processing units can have algorithms to calculate result outputs from, e.g., sensor input data. The processing units can have, e.g., digital memory 34 to store instructions for carrying out one or more assays, and to retain data from device sensors.

### Methods for Detecting Cancer-Associated Nucleic Acids

The methods for detecting the presence of cancer-associated nucleic acids can include obtaining a fluid sample of interest, applying the sample to a microfluidic cartridge so that it flows to come in contact with the sensor surface, applying an AC current to the sensor and measuring the impedance or frequency count from the sensor. The presence of hypomethylated and/or clustered methylation nucleic acids can be detected by, e.g., the presence of impedance or frequency rate of change or a change above a present value.

The fluid handling, control, and data acquisition for the methods can be provided by a combination of a microfluidic cartridge with a detector device (e.g., processor providing an AC current output to sensors in the cartridge). The processor can include software to analyze the sensor output data to provide a qualitative and/or quantitative result output. The detector device can be as described above in the Systems section, above.

Samples are typically biological samples. Often the samples are clinical samples, such as a body fluid or fluid obtained from cells or a tissue. Most commonly, the sample is a blood or biopsy sample. The nucleic acid can be, e.g., circulating cell-free nucleic acid and/or nucleic acids found in circulating cells. For example, an active tumor can experience apoptosis, releasing free nucleic acids into blood or lymph flows. Alternately, the tumors can shed whole cells into the blood stream. The methods are compatible with analysis of cells from tissues and organs. Typically, the tissue sample (e.g., biopsy) can be lysed (by sonication, detergents, freeze-thaw or the like), then the lysate can be applied directly to the cartridge, or solids can be removed (e.g., by centrifugation of filtration) before applying the tissue fluid. The cancer-associated nucleic acid of interest in the assays can be, e.g., DNA, or RNA.

In the methods, sample fluids are typically directed to flow through channels of the cartridges by capillary action to the detection electrodes. Optionally, fluid flows can be generated by application of a pressure differential to the cartridge. The fluids may flow through filters in the input port or downstream from the input port. The fluids may come into contact with timing gates, e.g., in embodiments wherein the assay has a rate or endpoint detection format. The fluids may come into contact with one or more reagents in a channel or chamber of the cartridge. For example, the fluid may contact a buffer composition setting the fluid at a desired pH and/or desired ionic strength, e.g., optimized to enhance resolution of hypomethylated/clustered nucleic acids over normal nucleic acids (e.g., by increasing the affinity of hypomethylated/clustered nucleic acids for the detection electrodes). Optionally, the sample can be exposed to a reagent that interacts with cysteines to make them more hydrophilic, or with methylated cytosines to make them more hydrophobic, thus modulating the aggregation and surface affinity differences.

Fluid flow can continue to the detection sensors (e.g., impedance or piezo sensors). Sensor surfaces can be positioned in the channel or in a chamber, e.g., at locations and with spacing to enhance sensitivity, precision, and/or range of impedance detection, depending on the particular assay format. The impedance detection electrode surfaces in microfluidic cartridges are typically spaced apart from each other with their surfaces in parallel planes.

On contact, the sample hypomethylated/clustered nucleic acids are adsorbed onto the sensor surfaces. The adsorbed nucleic acids can change the dielectric permittivity or mass of the sensor surfaces, resulting in a detectable change in the impedance or resonant frequency of the sensor. The change can be detectable, e.g., by a change in a resonant frequency of the electrode circuit, or by a change in the AC current in the circuit. In the present methods, hypomethylated/clustered nucleic acid detection is not based on prior art oxidation/reduction, imaging, or amperometric technologies.

The presence and/or quantity of hypomethylated/clustered nucleic acids in the sample fluid can be detected by monitoring the sensor impedance of vibration frequency. The fluid flow can be configured to stop when the fluid contacts the sensor surface or the fluid can continue flowing past the surface (e.g., into an efferent waste channel of chamber). A sensor output value can be determined at a particular time point or the rate of change in output can be monitored over a particular time frame. For example, the fluid can stop in a chamber in contact with the sensor surfaces and output can be measured after absorption has continued for from less than 1 second, to more than 60 seconds, from 3 seconds to 30 seconds, or about 10 seconds. In another example, the fluid can continue to flow past the sensor surfaces while nucleic acid adsorption (and sensor output) increases over time.

The presence of hypomethylated/clustered nucleic acids can be detected as, e.g., an impedance or frequency change over a predetermined threshold value or as a predetermined rate change. Optionally, the quantity of hypomethylated/clustered nucleic acids can be determined, e.g., based on an established regression curve, or comparison to negative controls and/or reference standards run on the same (multi-channel) cartridge at the same time.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Calibration of Devices to Select Cancer Cell Nucleic Acids

The devices for cancer nucleic acid analysis can be calibrated to distinguish or resolve hypomethylated nucleic acids from nucleic acids of normal tissue sources.

For example, representative human nucleic acids or those of a particular patient can be treated to prepare an array or gradient of nucleic acid samples with a range of methylation levels. The samples can be applied to a sensor adsorption surface of choice and the amount of adsorption noted for samples of each percent methylation. In a preferred embodiment, the surface is gold and the percent adsorption is measured by a piezo sensor or impedance sensor, though alternate methods of adsorption characterization can be used (e.g., electron microscopy or use of colloid gold color change). The range of methylation levels for standards can be prepared using methylation and/or demethylation enzymes, such as M.SssI CpG methyltransferase or oxidative demethylation enzymes.

The affinity and uniformity of adsorption can be influenced by the ionic strength and pH of the assay environment. Normal percent methylation can vary, e.g., depending on the sample source species. Normal human DNA is typically about 65 percent methylated (as determined by a methylation specific capture antibody—see Example 2, below). Methylation of cancer nucleic acids typically ranges from 30% to 50% or about 45%. A good rule of thumb is to optimize conditions for maximum selective adsorption of about 43% methylated nucleic acids.

To identify optimum selective conditions for adsorption of hypomethylated nucleic acids (e.g., from a cancer cell source), a methylation percent series of nucleic acid samples can be tested through a range of buffer conditions. For example, a percent methylation standard series can be adjusted to alternate sets of buffers, e.g., 200 mM, 300 mM, and 400 mM of a sodium salt, e.g., to determine which best provides the strongest signal for a particular sample type on a particular adsorption surface and/or, e.g., at a particular range of methylation of interest. An additional series around the favored buffer can further optimize the assay for the particular sample, sensitivity, precision, and hardware desired. Depending on the severity of the cancer and the nature of the sample, optimization can provide detection of cancer nucleic acid in cell free DNA of 5 ng or less, e.g., presenting 1% or less of nucleic acid from cancer cells.

### Example 2

### Calculation of Percent Methylation

Methylation analysis can be performed using, e.g., an Imprint™ Methylated DNA Quantification kit from Sigma Aldrich. This kit is essentially a sandwich assay in a 96-well format. Methylated nucleic acids are captured by a capture antibody on the well bottom. A secondary antibody with a reporter moiety is added before a wash and detection using a reporter reagent that develops a yellow coloration in the presence of specifically bound methylated nucleic acids.

The absorbance of the solutions in each well is measured at 450 nm using a plate reader. The global methylation level of the captured nucleic acids is calculated using following equation:

Methylation %=[(A_Sample−A₄₅₀ Blank)/(A₄₅₀ Methylated Control−A₄₅₀ Blank)]×100

### Example 3

### Materials used in the Practice of Methods Examples 4 to 6, Below

DNA Solution A was a genomic DNA solution from JURKAT cancer cells having a methylation level of 30% at concentration of 10 μg/mL in SSC 5× buffer with pH neutral. DNA Solution A was obtained by diluting CpG Methylated JURKAT Genomic DNA (15 ug, 0.1 mg/mL; Fisher Scientific) in SSC 5× buffer.

DNA Solution B was a DNA solution from Whole Genome Amplified (WGA) JURKAT DNA with methylation level of 0% at concentration of 10 μg/mL in SSC 5× buffer with pH neutral.

Whole Genome Amplification of JURKAT cancer cell DNA was prepared. In order to erase all the methylation marks in JURKAT DNA, we performed whole genome amplification on CpG Methylated Jurkat Genomic DNA (15 ug, 0.1 mg/mL) using REPLI-g Mini Kit (25) (Qiagen, Germantown Md. 20874) according to manufacturer instructions. The resultant WGA JURKAT DNA solution was 0.4 mg/mL as determined by standard fluorescence method, and had a methylation level of near 0% as determined using an Imprint Methylated DNA Quantification Kit (Sigma-Aldrich, Saint Louis) according to manufacturer instructions. Finally, DNA Solution B was obtained by diluting the WGA JURKAT DNA solution obtained from 1 above in SSC 5× buffer.

Instrument 1 was an Electrochemical Impedance Spectroscopy (EIS). All EIS measurements were performed using Interface 1010E Potentiostat/Galvanostat/ZRA (Gamry Instruments, Warminster, Pa. 18974).

Instrument 2 was a Quartz Crystal Microbalance (QCM). All QCM measurements were performed using eQCM 10M—Electrochemical Quartz Crystal Microbalance (Gamry Instruments, Warminster, Pa. 18974).

### Example 4

### Simple Setup for Methylation Analysis

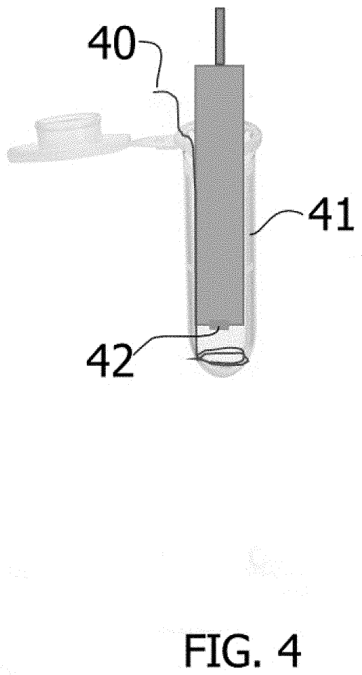

This Setup 1 used platinum wire and gold disk electrodes in 2 mL centrifuge tubes, as shown in FIG. 4.

A platinum wire 40 was curved at one end and placed in the bottom of a Eppendorf safe-lock tube 41 (2.0 mL, natural color, Cat #022363352, from Fisher Scientific, 300 Industry Drive, Pittsburgh, Pa. 15275). A gold disk electrode 42 (gold disk (3 mm in diameter) is molded into a 7 mm PEEK (polyetheretherketone) tube body (Gamry Instruments). The molded unit was placed in the Eppendorf tube 3 mm above the platinum wire end.

The above device was employed to provide electrochemical impedance spectroscopy (EIS) measurements. In order to find the optimal frequencies that would be most sensitive to the adsorption of DNA adsorption on the gold surface, 0.6 mL of DNA solution A was injected in Setup 1. EIS spectra were collected using Instrument 1 at different times (from 1 min to 10 min) after injection of DNA Solution A, through a frequency range from 1 Hz to 500,000 Hz at AC amplitude of 50 mV. It was indicated that the most sensitive frequency range was from 100 Hz to 100,000 Hz.

Note that before each use the gold disk electrode was cleaned by polishing with Alumina polishing powder (Gamry Instruments) followed by ultra-sonication with acetone and deionized water for 2 min.

In the present examples, we chose 1000 Hz to monitor the impedance change over the exposure time of DNA solution in contact with the gold electrode. 0.6 mL of DNA solution was injected into Setup 1 and the impedance was monitored using Instrument 1 at frequency of 1000 Hz for 10 min. The percentage change of the impedance for both DNA Solution A and DNA Solution B is summarized in Table 1. For control, the impedance change of SCC 5× buffer was also recorded.

TABLE 1
Impedance Change in Simple Setup 1

| | | | % Change of |
|---|---|---|---|
| | DNA | | Impedance at |
| | Methylation | | 10 min as |
| Solution | Level | Frequency | Compared to 0 min |
| DNA Solution A | 30% | 1000 Hz | 28% |
| DNA Solution B | 0% | 1000 Hz | 5% |
| SSC 5X Buffer | NA | 1000 Hz | 5% |

This data shows that AC impedance can be used to distinguish DNA's with higher methylation levels from lower ones.

### Example 5

### Microfluidic Electrochemical Impedance Spectroscopy (EIS)

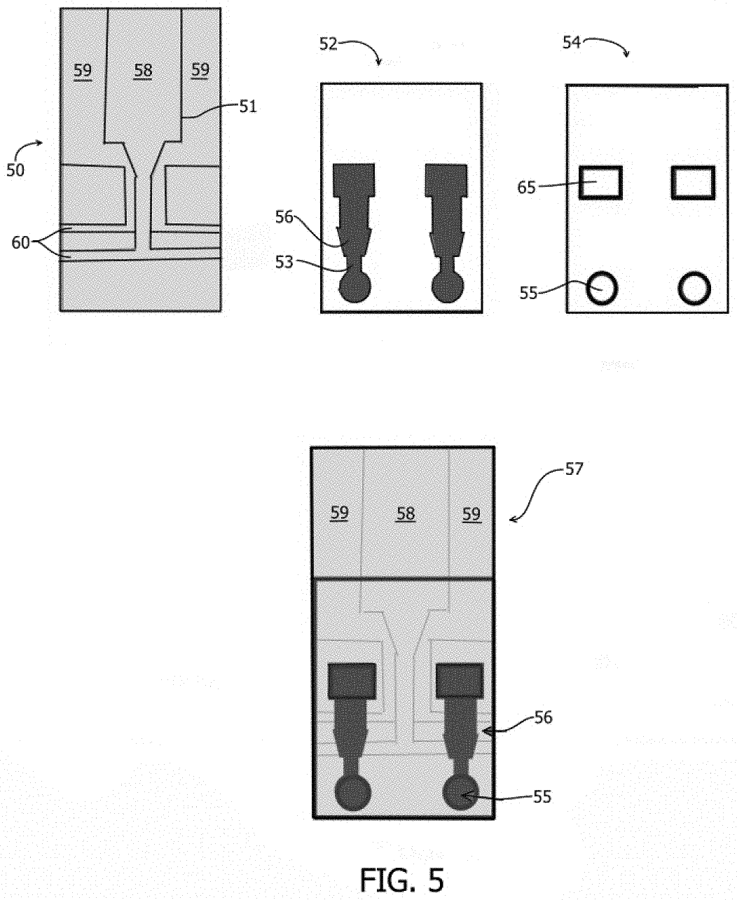

A gold strip cartridge comprised an electrode layer, a channel layer, and a cover layer, as shown in FIG. 5.

The electrode card 50 was made of gold sputtered onto a PET film with the gold thickness at 40 nm and the PET thickness at 254 μm (Materion Products). In order to form the electrodes, the gold film was placed on a KLIC-KUT MAXX digital cutter and traces of gold layer were removed by diamond knife along the conductor limiting lines 51, thereby delimiting conductivity to various contiguous regions of the film. The segregated electrodes can include a common input electrode 58 and separate test electrodes 59.

The pressure sensitive adhesive (PSA) card 52 is a of 80 μm thick plastic sheet with pressure sensitive adhesive on each side. In order to define the channels 53, the card was punched out along the shaded areas.

The cover card 54 was made of 170 μm PET film with a hydrophilic coating to facilitate the test solution capillary travel of aqueous solutions from the entry hole 55 to the test zone 56. Sample entry holes 55 (round areas) and vent holes 65 (rectangle areas) were punched out were punched out from the cover card 54.

An assembled gold strip cartridge 57 was formed by adhesively laminating the PSA card 50 onto the electrode card 52, then laminating the cover card 54 onto the other side of the PSA card.

EIS measurement with the gold strip cartridge 57 was used to determine impedance of adsorbed DNA samples. The single use cartridge had two PSA formed channels 53 and two pairs of gold thin film electrodes. One pair of electrodes 60 can be used a control and the other pair for test sample detection.

After a drop of DNA solution or control solution (˜5 μL) was added to respective entry holes 55, EIS spectra were collected using Instrument 1 from time 0 to 10 min in the frequency range from 1 Hz to 500,000 Hz at AC amplitude of 50 mV. It is indicated that the most sensitive frequency range for this configuration was from 100 Hz to 100,000 Hz.

In examples here, we chose 1000 Hz as the frequency to monitor the impedance change as a function of exposure time of DNA solution in the test zone. 5 μL of DNA solution was added into cartridge entry holes 55. The impedance across electrode pairs 60 was then monitored using Instrument 1 at frequency of 1000 Hz for 10 min. The percentage change of the impedance for both DNA Solution A (30% methylation) and DNA Solution B (0% methylation) is summarized in Table 2. For control, the impedance change of SCC 5× buffer was also recorded.

TABLE 2
Impedance Change in Microfluidic Cartridge

| | | | % Change of |
|---|---|---|---|
| | DNA | | impedance at |
| | Methylation | | 10 min as |
| Solution | Level | Frequency | compared to 0 min |
| DNA Solution A | 30% | 1000 Hz | 6% |
| DNA Solution B | 0% | 1000 Hz | 3% |
| SSC 5X Buffer | NA | 1000 Hz | −6% |

The decrease of the impedance for SSC 5× buffer may have been due to possible PSA swelling during the 10 min wait time, resulting in the increase of the electrode surface areas in the test zone. Such a phenomenon in this control would also have been experienced in the test channels. As compared to Example 4 above, the percent change of the impedance for DNA Solution A was smaller here. This might have been due to the design of the strip where the entire test zone was covered by gold film and the inactive gold surface might also adsorb DNA, e.g., reducing the amount of DNA to be adsorbed on electrodes. However, the difference in observed percent change between DNA solution B and A was still substantial and significant.

### Example 6

### Quartz Crystal Resonance Detection of Methylated DNA Levels

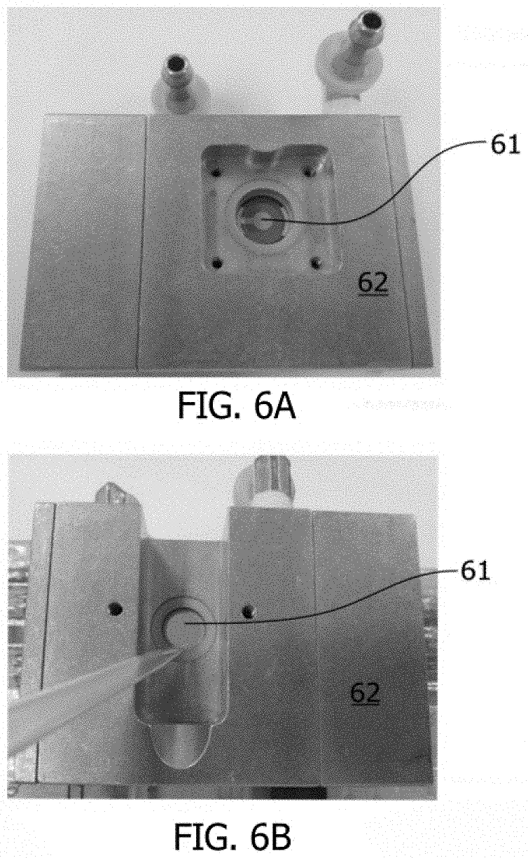

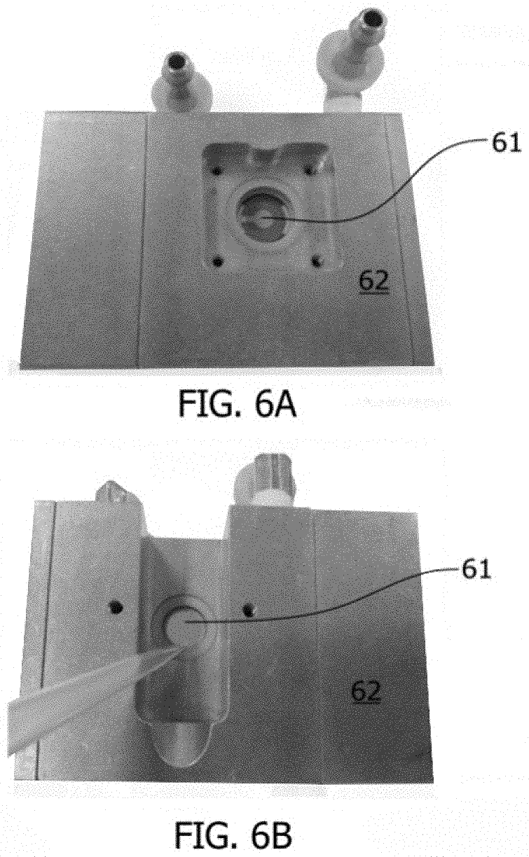

A 5 MHz Au quartz crystal wrap-around electrode was installed in temperature controlled eQCM cell kit (Gamry Instruments) according to manufacturer instructions. Specifically, the gold-coated crystal 61 was mounted in a window in the back of the temperature controlled cell kit 62, as shown in FIG. 6A. A BNC cable connector was mounted over the crystal to provide power and sensor communication. On the opposite side of the cell kit 62 and crystal 61, 0.2 mL of sample was applied to the gold surface, as shown in FIG. 6B. The surface was protected with a PEEK covering and the resonant frequency of the crystal monitored through the BNC cable.

Since it was difficult to clean, each of the 5 MHz Au quartz crystal wrap-around electrodes was used only once. After the 0.2 mL of the DNA solution was loaded on top of the gold surface, the series resonant frequency (Fs) was monitored as a function of time for 10 minutes. Table 3 summarizes the results.

TABLE 3
Methylation Levels by Monitoring Crystal Resonance

| | DNA | | |
|---|---|---|---|
| | Methylation | Initial | Change in Fs (Hz) |
| Solution | Level | Fs (MHz) | after 10 min |
| DNA Solution A | 30% | 4.9721 | −180 |
| DNA Solution B | 0% | 4.9711 | −12 |
| SSC 5X Buffer | NA | 4.9714 | −5 |

The DNA Solution A appeared to have more adsorption on the gold surface, resulting in larger scale in frequency drop than that of the DNA Solution B. DNA methylation levels are detectable according to their adsorption onto the gold surface and resultant change in the resonant frequency of the crystal. It is expected that this relationship can be interpreted to provide qualitative and quantitative DNA methylation results.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A cancer cell nucleic acid detection system comprising a cartridge and a detector device:
the cartridge comprising a sample input port leading to paired detector electrodes through a first microfluidic channel; the electrodes comprising one or more surfaces adapted to adsorb methylated nucleic acids; and conductors leading from the detector electrodes to contact pads on an outer surface of the cartridge;
the detector device comprising an AC power source and an impedance detector in contact with the cartridge contact pads and configured to measure impedance between the electrodes;
whereby hypomethylated nucleic acids associated with the cancer cell adsorb onto the one or more surfaces of the detector electrodes more uniformly or with higher affinity than nucleic acids of non-cancer cells, resulting in a higher impedance detected for samples having cancer cell nucleic acids than for normal samples not containing cancer cell nucleic acids.

2. The system of claim 1, wherein the cartridge is a microfluidic cartridge.

3. The system of claim 1, wherein the electrodes comprise a gold or platinum surface.

4-9. (canceled)

10. The system of claim 1, further comprising a second channel in the cartridge configured to provide a confirmatory or control assay.

11. The system of claim 1, adapted to detect cancer cells with hypomethylated nucleic acids having methylation values from 30% to 50% or methylation clustering with more than half the methylated cysteines represented in 20% or less of the nucleic acid.

12. The system of claim 1, wherein the detector is configured to confirm the presence of the cancer cell nucleic acid when the detected impedance is above an established impedance cut-off value.

13. The system of claim 1, wherein the system is configured so that the hypomethylated nucleic acids adsorb on the detection electrode surface without a prior aggregation step in solution.

14. A method of detecting the presence of a cancer in a patient, the method comprising:
providing a microfluidic cartridge comprising a sample input port, a first microfluidic channel and a pair of electrodes, wherein a fluid sample applied to the input port flows by capillary action through the channel to the pair of electrodes;
providing an AC current power supply and an impedance detector in electrical contact with the electrodes;
applying a patient biological fluid sample to the input port wherefrom the fluid flows through the channel to contact the electrode pair;
applying an AC current across the electrode pair from the power supply and detecting an impedance between the paired electrodes; and,
confirming the presence of nucleic acids from a cancer when the detected impedance is higher for the sample than for a sample without nucleic acids from a cancer.

15-17. (canceled)

18. The method of claim 17, further comprising determining the presence of a hypomethylated cancer cell nucleic acid when the measured impedance increases by 20 percent within 600 seconds of contact of the electrode pair with the fluid sample.

19. The method of claim 14, further comprising providing a second channel in the cartridge with a second electrode pair, flowing a reference fluid sample to the second electrode pair, and determining the presence or quantity of hypomethylated nucleic acids from a cancer by comparison to a reference impedance at the second electrode pair.

20. (canceled)

21. The method of claim 14, further comprising determining a cut-off impedance value for the cartridge wherein impedance values above the cut-off indicate the presence of hypomethylated cancer cell nucleic acids.

22. The method of claim 21, wherein the cut-off value is determined empirically by review of reference samples of known hypomethylation character.

23. The method of claim 14, wherein said detecting is by other than by redox potential measurement, amperometry, or imaging of surface pattern.

24. (canceled)

25. The method of claim 14, further comprising confirming the presence of cancer cell nucleic acid when the percent methylation is within the range from 30% to 50% or when more than 50% of the nucleic acid methylations in less than 20% of the nucleic acid genes.

26. (canceled)

27. The method of claim 14, further comprising configuring the fluid sample pH or ionic strength to provide optimal adsorption for hypomethylated nucleic acids of interest.

28. A cancer cell nucleic acid detection system comprising a cartridge and a detector device:
the cartridge comprising a sample input port leading through a first microfluidic channel to a piezoelectric crystal sensor having a gold surface adapted to adsorb nucleic acids; and conductors leading from the sensor to one or more contact pads on an outer surface of the cartridge;
the detector device comprising an oscillator circuit output configured to stimulate a base frequency in the crystal, and comprising a frequency counter detector in contact with the cartridge contact pads and configured to measure a frequency of crystal vibrations;
wherein hypomethylated nucleic acids associated with the cancer cell adsorb onto the crystal gold surface with higher uniformity or affinity than nucleic acids of non-cancer cells; and,
whereby the presence of the hypomethylated nucleic acids is detected by the frequency counter as a lower vibration frequency from the crystal.

29. The system of claim 28, wherein the cartridge is a microfluidic cartridge.

30-31. (canceled)

32. The system of claim 28, configured to confirm the presence of a 30% to 50% methylated nucleic acid when a 10 Hz to 10 KHz drop is detected in series or parallel frequency of the crystal oscillation.

33-36. (canceled)

37. The system of claim 28, further comprising a second channel in the cartridge configured to provide a confirmatory or control assay.

38. The system of claim 28, adapted to detect cancer cell nucleic acids having a methylation values from 30% to 50% or methylation clustering with more than half the methylated cysteines represented in 20% or less of the nucleic acid.

39-43. (canceled)
